(19) United States
(12) Patent Application Publication
(10) Pub. No.: US 2025/0171403 A1
(43) Pub. Date: May 29, 2025

(54) CABOZANTINIB SALT WITH L-(+)-TARTARIC ACID AND SOLID FORMS THEREOF

(71) Applicant: Synthon B.V., Nijmegen (NL)

(72) Inventors: Bohumil DYMACEK, Blansko (CZ); Katerina JELINKOVA, Blansko (CZ)

(21) Appl. No.: 18/842,577

(22) PCT Filed: Feb. 28, 2023
(86) PCT No.: PCT/EP2023/054897

§ 371 Date: Aug. 29, 2024

(30) Foreign Application Priority Data
Mar. 1, 2022 (EP) 22159429.4

(51) Int. Cl. C07D 215/22

(52) U.S. Cl. CPC C07D 215/22

(57) ABSTRACT

The invention relates to solid forms of Cabozantinib, compound of formula (1), salt with L-(+)-tartaric acid and processes for preparation thereof,

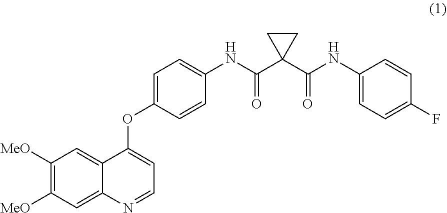 [SMILES: COc1cc2nccc(Oc3ccc(NC(=O)C4(C(=O)Nc5ccc(F)cc5)CC4)cc3)c2cc1OC]

The invention relates to solid forms of Cabozantinib salt with L-(+)-tartaric acid and processes for preparation thereof.

### BACKGROUND OF THE PRESENT INVENTION

This invention relates to Cabozantinib salt with L-(+)-tartaric acid, solid forms thereof and processes for preparation thereof.

Cabozantinib, N-[4-(6,7-Dimethoxyquinolin-4-yloxy)phenyl]-N′-(4-fluorophenyl)cyclopropane-1,1-dicarboxamide, compound of formula (1),

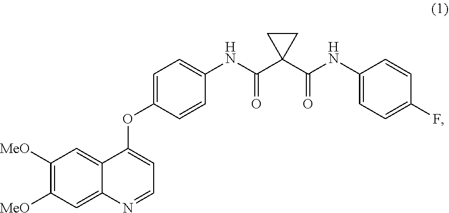 [SMILES: COc1cc2nccc(Oc3ccc(NC(=O)C4(C(=O)Nc5ccc(F)cc5)CC4)cc3)c2cc1OC]

is an orally bioavailable antineoplastic agent approved for the treatment of unresectable, locally advanced or metastatic medullary thyroid cancer (MTC). Cabozantinib is marketed in form of L-malate salt.

Cabozantinib was first disclosed in WO2005030140 application. Malate salt of Cabozantinib was disclosed in WO2010083414 application. WO2010083414 discloses that tartrate salt of Cabozantinib has low crystallinity, low solubility and is hygroscopic. WO2018218233 application described other Cabozantinib salts and solid forms thereof.

Cabozantinib and its salts described in the prior art are poorly soluble. There is still a need to prepare Cabozantinib salts and solid forms thereof having improved solubility, crystallinity and stability in comparison with Cabozantinib salts disclosed in the prior art.

### BRIEF DESCRIPTION OF THE INVENTION

The invention relates to solid forms of Cabozantinib salt with L-(+)-tartaric acid and processes for preparation thereof.

### BRIEF DESCRIPTION OF THE DRAWINGS

FIG. 1 depicts the X-Ray Powder Diffractogram (XRPD) of Cabozantinib salt with L-(+)-tartaric acid, Form 1, prepared according to Example 1 or Example 2.

FIG. 2 depicts the DSC pattern of Cabozantinib salt with L-(+)-tartaric acid, Form 1, prepared according to Example 1 or Example 2.

FIG. 3 depicts the TGA pattern of Cabozantinib salt with L-(+)-tartaric acid, Form 1, prepared according to Example 1 or Example 2.

FIG. 4 depicts the X-Ray Powder Diffractogram (XRPD) of Cabozantinib salt with L-(+)-tartaric acid, Form 2, prepared according to Example 3 or Example 4 or Example 9.

FIG. 5 depicts the DSC pattern of Cabozantinib salt with L-(+)-tartaric acid, Form 2, prepared according to Example 3 or Example 4 or Example 9.

FIG. 6 depicts the TGA pattern of Cabozantinib salt with L-(+)-tartaric acid, Form 2, prepared according to Example 3 or Example 4 or Example 9.

FIG. 7 depicts the X-Ray Powder Diffractogram (XRPD) of Cabozantinib salt with L-(+)-tartaric acid, Form 3, prepared according to Example 5 or Example 6 or Example 7.

FIG. 8 depicts the DSC pattern of Cabozantinib salt with L-(+)-tartaric acid, Form 3, prepared according to Example 5 or Example 6 or Example 7.

FIG. 9 depicts the TGA pattern of Cabozantinib salt with L-(+)-tartaric acid, Form 3, prepared according to Example 5 or Example 6 or Example 7.

FIG. 10 depicts the X-Ray Powder Diffractogram (XRPD) of Cabozantinib salt with L-(+)-tartaric acid, Form 4, prepared according to Example 8.

FIG. 11 depicts the DSC pattern of Cabozantinib salt with L-(+)-tartaric acid, Form 4, prepared according to Example 8.

FIG. 12 depicts the TGA pattern of Cabozantinib salt with L-(+)-tartaric acid, Form 4, prepared according to Example 8.

FIG. 13 depicts NMR pattern of Cabozantinib salt with L-(+)-tartaric acid, Form 1, prepared according to Example 1 or Example 2.

FIG. 14 depicts NMR pattern Cabozantinib salt with L-(+)-tartaric acid, Form 2, prepared according to Example 3 or Example 4.

FIG. 15 depicts NMR pattern Cabozantinib salt with L-(+)-tartaric acid, Form 3, prepared according to Example 5 or Example 6 or Example 7.

FIG. 16 depicts NMR pattern Cabozantinib salt with L-(+)-tartaric acid, Form 4, prepared according to Example 8.

FIG. 17 depicts the X-Ray Powder Diffractogram (XRPD) of Cabozantinib salt with L-(+)-tartaric acid, Form 2A, prepared according to Example 9.

FIG. 18 depicts the DSC pattern of Cabozantinib salt with L-(+)-tartaric acid, Form 2A, prepared according to Example 9.

FIG. 19 depicts the TGA pattern of Cabozantinib salt with L-(+)-tartaric acid, Form 2A, prepared according to Example 9.

### DETAILED DESCRIPTION OF THE INVENTION

The presented invention relates to Cabozantinib salt with L-(+)-tartaric acid, solid forms thereof (Form 1, Form 2, Form 3, Form 4) and to processes for preparing thereof. Contrary to the teaching of WO2010083414 application, Cabozantinib salts with L-(+)-tartaric acid according to the presented invention show good crystallinity, improved solubility and low hygroscopicity.

The Form 1 of Cabozantinib salt with L-(+)-tartaric acid can be characterized by XRPD pattern having 2θ values 3.9°, 13.5°, 14.8° and 23.1° degrees 2 theta (±0.2 degrees 2 theta). The solid Form 1 can be also characterized by XRPD pattern having 2θ values 3.9°, 6.7°, 9.2°, 13.5°, 14.8°, 15.5° and 23.1° degrees 2 theta (±0.2 degrees 2 theta). The solid form can be further characterized by XRPD pattern described in the following table:

| Angle (2θ) | Intensity (%) |
|---|---|
| 3.8 | 26.7 |
| 4.4 | 7.3 |
| 5.7 | 6.8 |
| 6.7 | 19.6 |
| 7.6 | 18.6 |
| 8.6 | 7.5 |
| 9.1 | 34.2 |
| 10.1 | 7.2 |
| 11.1 | 9.0 |
| 11.5 | 18.6 |
| 12.9 | 6.5 |
| 13.5 | 100.0 |
| 13.8 | 27.7 |
| 14.2 | 9.9 |
| 14.8 | 38.7 |
| 15.5 | 28.2 |
| 16.3 | 8.8 |
| 16.7 | 12.8 |
| 17.2 | 13.3 |
| 17.9 | 12.0 |
| 18.4 | 13.3 |
| 18.9 | 14.7 |
| 19.4 | 21.4 |
| 19.9 | 23.3 |
| 20.5 | 18.5 |
| 20.8 | 18.9 |
| 21.3 | 19.4 |
| 21.5 | 22.7 |
| 22.1 | 13.3 |
| 22.6 | 32.8 |
| 23.1 | 38.5 |
| 23.6 | 21.4 |
| 23.9 | 17.1 |
| 24.5 | 20.0 |
| 24.9 | 22.5 |
| 25.4 | 32.4 |
| 25.9 | 32.2 |
| 27.2 | 30.4 |
| 27.9 | 27.5 |
| 28.5 | 13.5 |
| 29.7 | 8.8 |
| 30.2 | 6.9 |
| 30.9 | 9.0 |
| 31.7 | 8.6 |
| 32.4 | 8.3 |
| 32.7 | 7.4 |
| 33.3 | 8.7 |
| 34.6 | 6.8 |

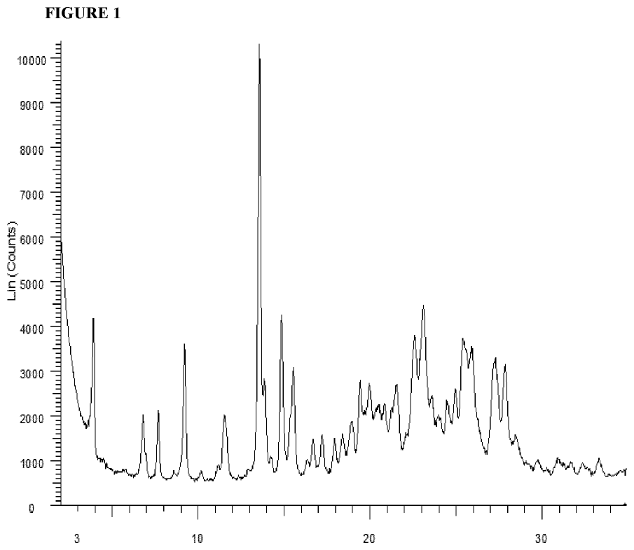

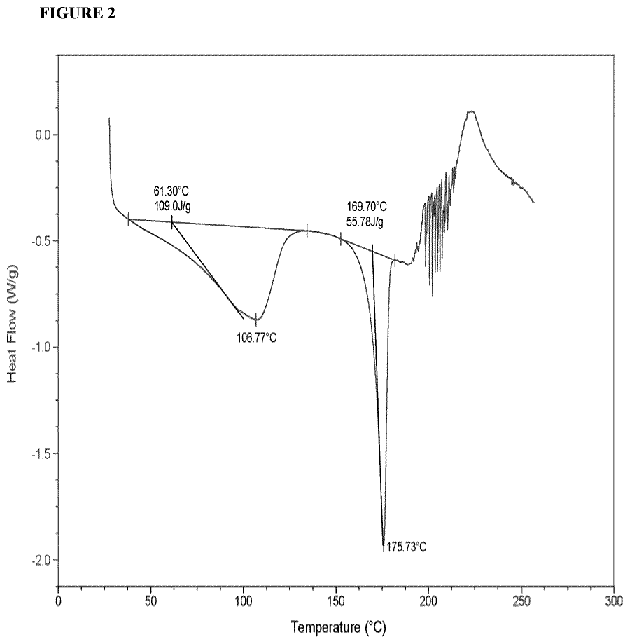

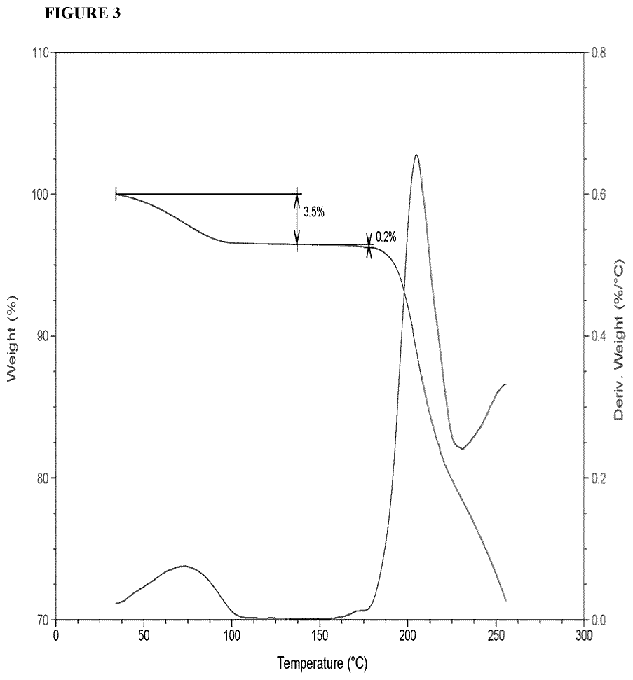

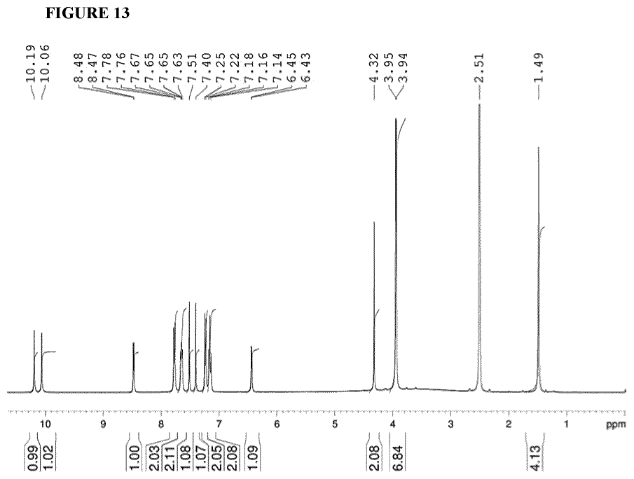

The solid Form 1 can be also characterized by XRPD pattern depicted in FIG. 1. The solid form can be further characterized by DSC pattern depicted in FIG. 2. The solid form can be further characterized by TGA pattern depicted in FIG. 3. The solid form can be also characterized by NMR pattern depicted in FIG. 13.

The solid Form 1 of Cabozantinib salt with L-(+)-tartaric acid can be prepared by a process comprising:
a. Mixing Cabozantinib with a mixture of tetrahydrofurane and 2.5-10% water (volume %) at a temperature between 55° C. and 65° C.;
b. Cooling the mixture at a temperature between 35° C. and 45° C.;
c. Adding L-(+)-tartaric acid;
d. Cooling the mixture to a temperature between 20° C. and 25° C.;
e. Isolating the solid form.

The concentration of Cabozantinib in the mixture tetrahydrofurane and water can be between 0.08 g/mg and 1.3 g/ml. 2.5-10% water (volume %) of water added to tetrahydrofurane significantly improve the solubility of Cabozantinib in the mixture. The molar ratio between Cabozantinib and L-(+)-tartaric acid can be between 1:1 and 1:1.2. Cabozantinib is mixed with the mixture of tetrahydrofurane and water at a temperature between 55° C. and 65° C. The mixture is cooled to a temperature between 35° C. and 45° C. To the mixture L-(+)-tartaric acid is added. L-(+)-tartaric acid can be added as solid or can be added as a solution of L-(+)-tartaric acid in a suitable solvent, for example tetrahydrofurane. The mixture is cooled to a temperature between 20° C. and 25° C. and stirred at this temperature for between 15 and 30 hours to obtain a suspension. The suspension is filtered off and dried, optionally under vacuum (1-10 kPa) at a temperature between 25° C. and 45° C. for between 15 and 30 hours to provide solid Form 1 of Cabozantinib salt with L-(+)-tartaric acid.

The solid Form 1 of Cabozantinib salt with L-(+)-tartaric acid can be also prepared by a process comprising:
a. Mixing Cabozantinib with a mixture of tetrahydrofurane, ethyl acetate and 1.5-10% water (volume %) preferably at a temperature between 55° C. and 65° C.;
b. Cooling the mixture preferably to a temperature between 35° C. and 45° C.;
c. Adding L-(+)-tartaric acid;
d. Cooling the mixture to a temperature between 20° C. and 25° C.;
e. Isolating the solid form.

The volume ratio between tetrahydrofurane and ethyl acetate can be between 1:1 and 1:1.2. The molar ratio between Cabozantinib and L-(+)-tartaric acid can be between 1:1 and 1:1.2. The concentration of Cabozantinib in the mixture tetrahydrofurane, ethyl acetate, water can be between 0.03 g/ml and 0.07 g/ml. 1.5-10% water (volume %) of water added to a mixture of tetrahydrofurane and ethyl acetate significantly improve the solubility of Cabozantinib in the mixture. To the mixture of tetrahydrofurane, ethyl acetate and water Cabozantinib is added preferably at a temperature between 55° C. and 65° C. To the mixture L-(+)-tartaric acid is added. L-(+)-tartaric acid can be added either as a solid or as a solution in a suitable solvent.

The mixture is cooled preferably to a temperature between 20° C. and 25° C. and stirred at this temperature for between 8 and 15 hours to obtain a suspension. The suspension is filtered off and obtained solid can be optionally dried.

The solid Form 2 of Cabozantinib salt with L-(+)-tartaric acid can be characterized by XRPD pattern having 2θ values 12.2°, 19.4°, 22.2° and 23° degrees 2 theta (±0.2 degrees 2 theta). The solid Form 2 can be also characterized by XRPD pattern having 2θ values 4.4°, 8.5°, 12.2°, 14.0°, 19.4°, 22.2° and 23.0° degrees 2 theta (±0.2 degrees 2 theta). The solid form can be further characterized by XRPD pattern described in the following table:

| Angle (2θ) | Intensity (%) |
|---|---|
| 4.4 | 34.9 |
| 6.0 | 10.9 |
| 8.2 | 14.5 |
| 8.5 | 41.1 |
| 8.7 | 31.5 |
| 11.2 | 15.4 |
| 12.0 | 62.4 |
| 12.2 | 100.0 |
| 14.0 | 38.8 |
| 14.4 | 28.1 |
| 14.9 | 14.0 |
| 16.1 | 21.6 |
| 16.4 | 31.8 |
| 17.0 | 38.7 |
| 17.5 | 33.4 |
| 17.9 | 18.1 |
| 19.4 | 55.1 |
| 19.9 | 31.5 |
| 20.8 | 32.4 |
| 21.4 | 47.7 |
| 21.8 | 31.2 |
| 22.2 | 60.4 |
| 23.0 | 53.5 |
| 23.7 | 53.5 |
| 24.5 | 73.3 |
| 24.8 | 53.8 |
| 25.7 | 55.3 |
| 26.0 | 48.2 |
| 26.8 | 49.8 |
| 27.2 | 62.9 |
| 27.7 | 41.7 |
| 28.4 | 33.8 |
| 29.0 | 35.2 |
| 30.2 | 25.4 |
| 31.9 | 21.4 |
| 33.2 | 22.5 |
| 34.0 | 21.6 |

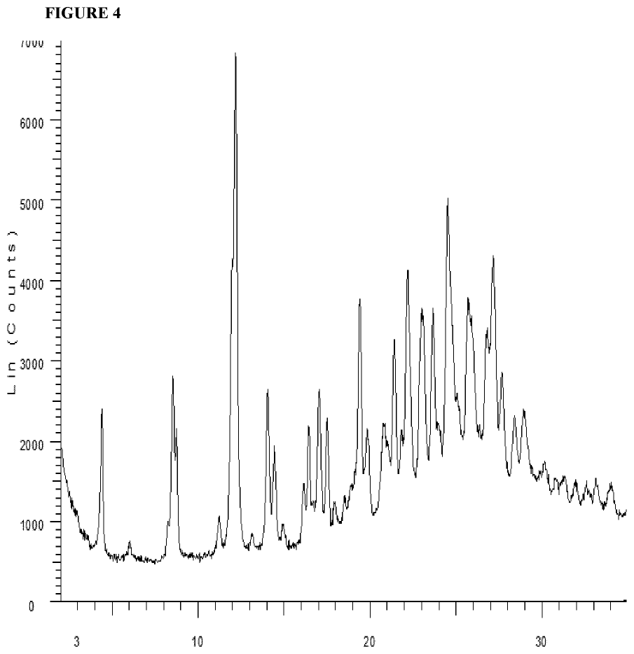

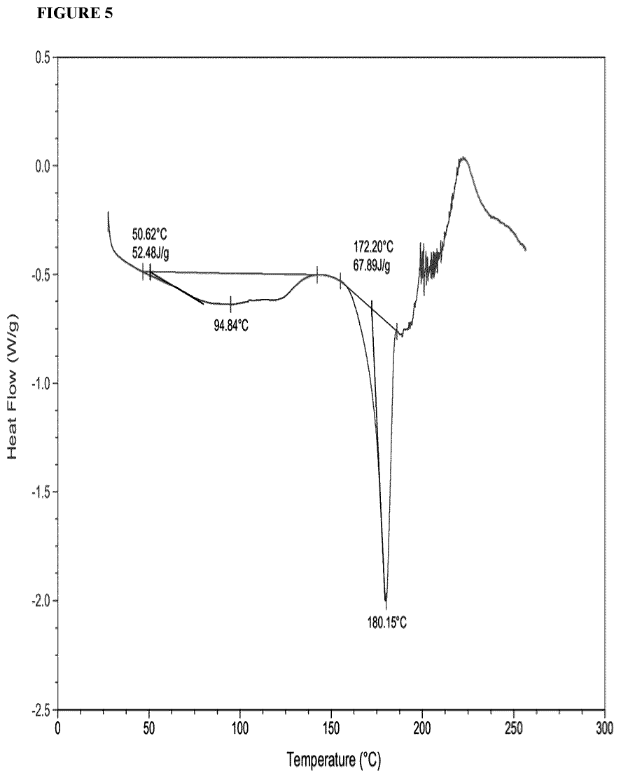

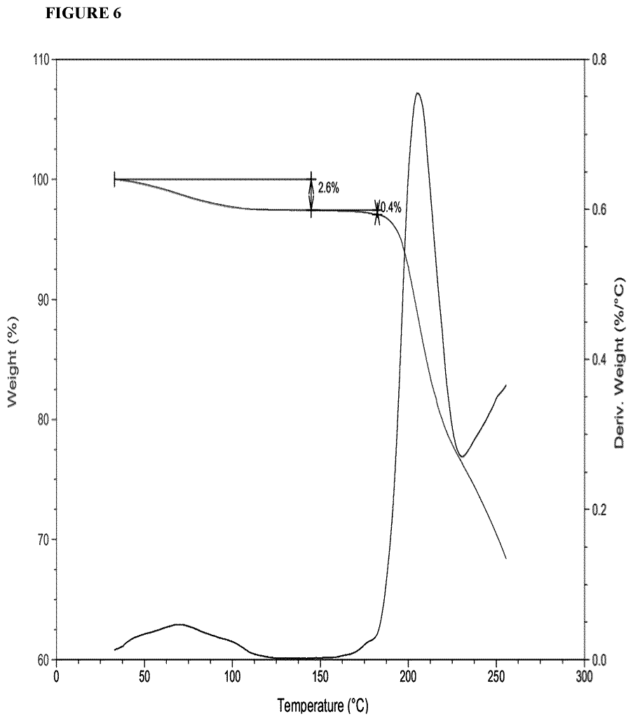

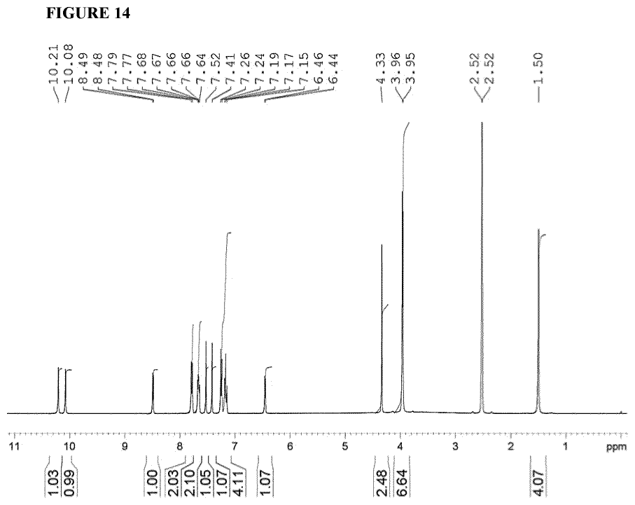

The solid Form 2 can be also characterized by XRPD pattern depicted in FIG. 4. The solid form can be further characterized by DSC pattern depicted in FIG. 5. The solid form can be further characterized by TGA pattern depicted in FIG. 6. The solid form can be also characterized by NMR pattern depicted in FIG. 14.

The solid Form 2 of Cabozantinib salt with L-(+)-tartaric acid can be prepared by a process comprising:
a. Mixing Cabozantinib with a mixture of tetrahydrofurane and methanol;
b. Adding L-(+)-tartaric acid;
c. Cooling the mixture;
d. Isolating the solid form.

The volume ratio between tetrahydrofurane and methanol can be between 1.1:1 and 1.4:1. The molar ratio between Cabozantinib and L-(+)-tartaric acid can be between 1:1 and 1:1.6, preferably it is between 1:1 and 1:1.2. The concentration of Cabozantinib in the mixture of tetrahydrofurane and methanol can be between 0.06 g/ml and 0.1 g/ml. Cabozantinib is mixed with the mixture of tetrahydrofurane and methanol preferably at a temperature between 60° C. and 70° C. To the mixture L-(+)-tartaric acid is added. L-(+)-tartaric acid can be added either as a solid or as a solution in a suitable solvent.

The mixture is cooled preferably to a temperature between 20° C. and 25° C. and stirred at this temperature for between 5 and 10 hours to obtain a suspension. The suspension is filtered off and obtain solid is optionally dried.

The solid Form 2 of Cabozantinib salt with L-(+)-tartaric acid can be prepared by a process comprising:
a. Mixing Cabozantinib with a mixture of tetrahydrofurane, toluene and 2.5-10% (vol %) of water;
b. Adding L-(+)-tartaric acid;
c. Cooling the mixture;
d. Isolating the solid form.

The volume ratio between tetrahydrofurane and toluene can be between 1:1 and 1:1.2. The concentration of Cabozantinib in the mixture of tetrahydrofurane, toluene and water can be between 0.06 g/ml and 0.1 g/ml. 2.5-10% water (volume %) of water added to a mixture of tetrahydrofurane and toluene significantly improve the solubility of Cabozantinib in the mixture. The molar ratio between Cabozantinib and L-(+)-tartaric acid can be between 1:1 and 1:1.6, preferably between 1:1 and 1:1.2. Cabozantinib is mixed with the mixture of tetrahydrofurane, toluene and water preferably at a temperature between 55° C. and 65° C. To the mixture L-(+)-tartaric acid is added. L-(+)-tartaric acid can be added either as a solid or as a solution in a suitable solvent. The mixture is cooled preferably to a temperature between 20° C. and 25° C. and stirred at this temperature for between 8 and 15 hours to provide a suspension. The suspension is filtered off and obtained solid is dried on air for between 8 and 15 hours.

The solid Form 2 can be also prepared by drying Cabozantinib salt with L-(+)-tartaric acid solid Form 2A preferably at a temperature between 60° C. and 90° C. for between 3 and 10 hours.

The solid Form 3 of Cabozantinib salt with L-(+)-tartaric acid can be characterized by XRPD pattern having 2θ values 10.2°, 13.5°, 15.4° and 22.8° degrees 2 theta (±0.2 degrees 2 theta). The solid Form 3 can be also characterized by XRPD pattern having 2θ values 3.9°, 8.4°, 10.2°, 12.8°, 13.5°, 15.4°, 22.8° and 25.9° degrees 2 theta (±0.2 degrees 2 theta). The solid form can be further characterized by XRPD pattern described in the following table:

| Angle (2θ) | Intensity (%) |
|---|---|
| 3.9 | 27.0 |
| 6.8 | 23.5 |
| 7.8 | 10.6 |
| 8.4 | 26.2 |
| 10.2 | 33.6 |
| 10.8 | 7.8 |
| 12.0 | 8.6 |
| 12.7 | 25.0 |
| 13.5 | 100.0 |
| 15.4 | 59.0 |
| 15.6 | 17.3 |
| 15.8 | 14.3 |
| 16.8 | 13.8 |
| 18.3 | 15.2 |
| 18.5 | 33.3 |
| 19.3 | 13.2 |
| 19.6 | 12.1 |
| 19.9 | 7.8 |
| 20.5 | 22.9 |
| 20.9 | 27.2 |
| 21.3 | 12.0 |
| 21.8 | 14.4 |
| 22.1 | 20.8 |
| 22.8 | 63.2 |
| 23.2 | 42.0 |
| 23.5 | 25.9 |
| 24.1 | 10.7 |
| 24.8 | 12.9 |
| 25.0 | 15.9 |
| 25.2 | 16.9 |
| 25.9 | 65.3 |
| 26.2 | 23.5 |
| 27.0 | 12.2 |
| 27.3 | 28.5 |
| 27.9 | 6.7 |
| 28.1 | 6.7 |
| 28.6 | 7.4 |
| 29.1 | 14.7 |
| 29.3 | 9.0 |
| 30.0 | 11.6 |
| 30.8 | 8.8 |
| 31.9 | 9.3 |
| 32.5 | 4.8 |
| 32.9 | 8.7 |
| 33.1 | 8.1 |
| 33.5 | 7.2 |
| 33.6 | 7.6 |
| 34.1 | 6.6 |

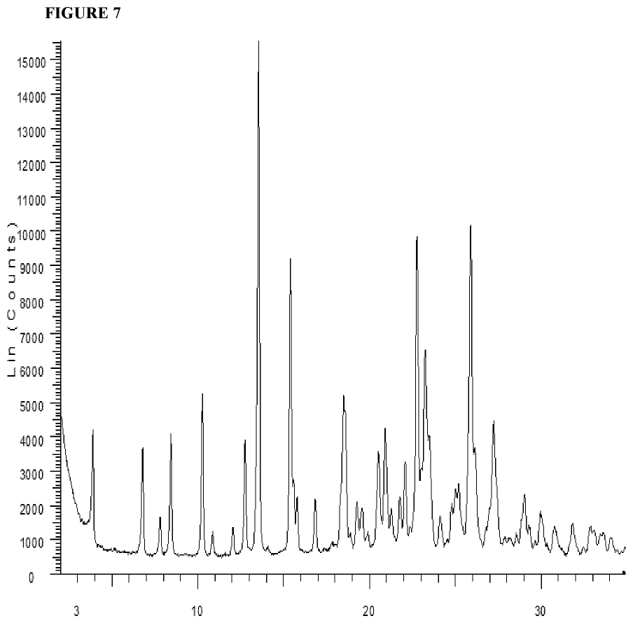

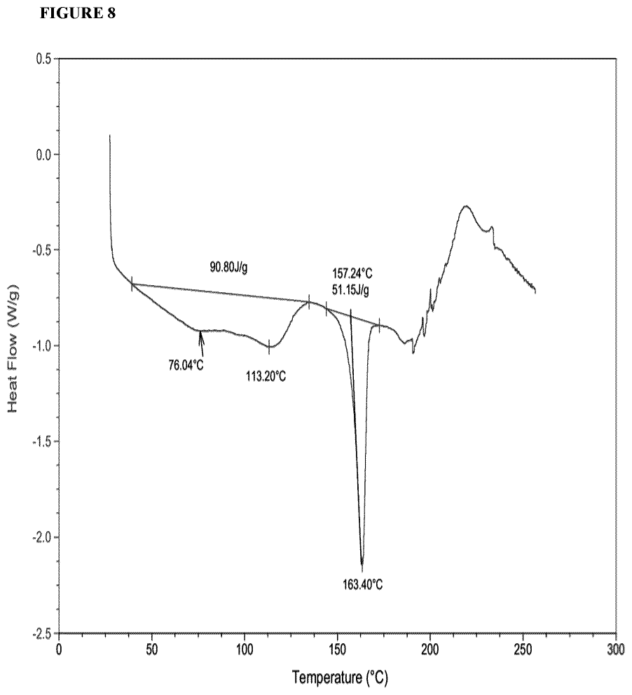

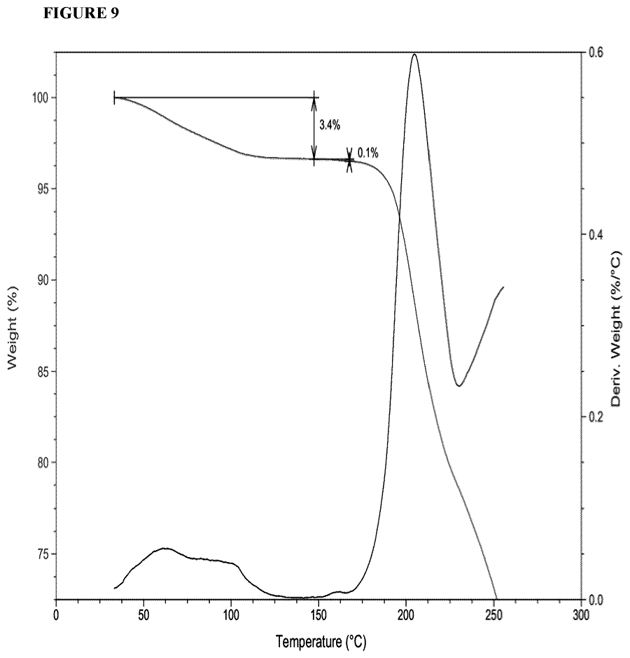

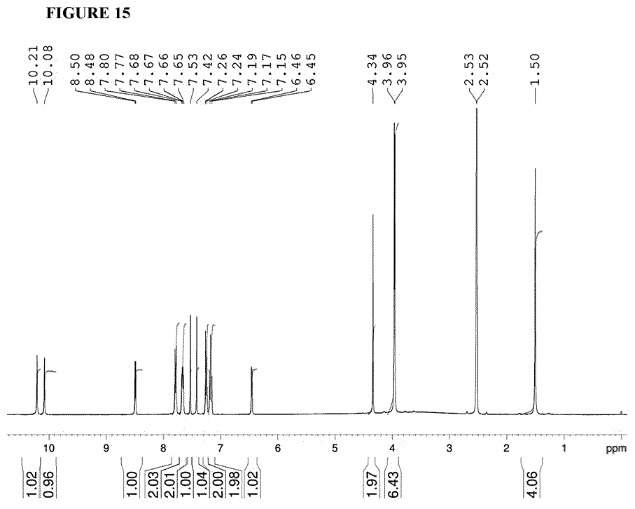

The solid Form 3 can be also characterized by XRPD pattern depicted in FIG. 7. The solid form can be further characterized by DSC pattern depicted in FIG. 8. The solid form can be further characterized by TGA pattern depicted in FIG. 9. The solid form can be also characterized by NMR pattern depicted in FIG. 15.

The solid Form 3 of Cabozantinib salt with L-(+)-tartaric acid can be prepared by a process comprising:
a. Mixing Cabozantinib with a mixture of tetrahydrofurane and 2.5-10% water (volume %) preferably at a temperature between 55° C. and 65° C.;
b. Adding L-(+)-tartaric acid;
c. Cooling the mixture preferably to a temperature between 20° C. and 25° C.;
d. Isolating the solid form.

The concentration of Cabozantinib in the mixture of tetrahydrofurane and water can be between 0.08 g/ml and 0.11 g/ml. 2.5-10% water (volume %) of water added to tetrahydrofurane significantly improve the solubility of Cabozantinib in the mixture. The molar ratio between Cabozantinib and L-(+)-tartaric acid can be between 1:1 and 1:1.2. Cabozantinib is mixed with the mixture of tetrahydrofurane and water preferably at a temperature between 55° C. and 65° C. To the mixture L-(+)-tartaric acid is added. L-(+)-tartaric acid can be added either as a solid or as a solution in a suitable solvent. The mixture is cooled preferably to a temperature between 20° C. and 25° C. and stirred at this temperature for between 8 and 15 hours to provide a suspension. The suspension is filtered off and obtained solid can be optionally dried.

The solid Form 3 of Cabozantinib salt with L-(+)-tartaric acid can be prepared by a process comprising:
a. Mixing Cabozantinib with a mixture of tetrahydrofurane, acetone and 2.5-10% water (volume %) preferably at a temperature between 55° C. and 65° C.;
b. Adding L-(+)-tartaric acid;
c. Cooling the mixture preferably to a temperature between 20° C. and 25° C.;
d. Isolating the solid form.

The concentration of Cabozantinib in the mixture of tetrahydrofurane, acetone and water can be between 0.07 g/ml and 0.11 g/ml. 2.5-10% water (volume %) of water added to a mixture of tetrahydrofurane and acetone significantly improve the solubility of Cabozantinib in the mixture. The molar ratio between Cabozantinib and L-(+)-tartaric acid can be between 1:1 and 1:1.6, preferably between 1:1 and 1:1.2. Cabozantinib is mixed with the mixture of tetrahydrofurane, acetone and water preferably at a temperature between 55° C. and 65° C. To the mixture L-(+)-tartaric acid is added. L-(+)-tartaric acid can be added either as a solid or as a solution in a suitable solvent. The mixture is cooled preferably to a temperature between 20° C. and 25° C. and stirred at this temperature for between 8 and 15 hours to provide a suspension. The suspension is filtered off and obtained solid can be optionally dried.

The solid Form 3 of Cabozantinib salt with L-(+)-tartaric acid can be also prepared by a process comprising:
a. Mixing Cabozantinib with a mixture of tetrahydrofurane, ethanol and 1.5-10% water (volume %) preferably at a temperature between 55° C. and 65° C.;
b. Adding L-(+)-tartaric acid;
c. Cooling the mixture preferably to a temperature between 20° C. and 25° C.;
d. Isolating the solid form.

The concentration of Cabozantinib in the mixture of tetrahydrofurane, ethanol and water can be between 0.07 g/ml and 0.11 g/ml. The volume ratio between tetrahydrofurane and ethanol can be between 1:1 and 1.2:1. 2.5-10% water (volume %) of water added to a mixture of tetrahydrofurane and ethanol significantly improve the solubility of Cabozantinib in the mixture. The molar ratio between Cabozantinib and L-(+)-tartaric acid can be between 1:1 and 1:1.6, preferably between 1:1 and 1:1.2. Cabozantinib is mixed with the mixture of tetrahydrofurane, ethanol and water preferably at a temperature between 55° C. and 65° C. To the mixture L-(+)-tartaric acid is added. L-(+)-tartaric acid can be added either as a solid or as a solution in a suitable solvent. The mixture is stirred at a temperature between 55° C. and 65° C. for between 20 and 60 minutes. The mixture is cooled preferably to a temperature between 20° C. and 25° C. and stirred at this temperature for between 8 and 15 hours to provide a suspension. The suspension is filtered off and obtained solid can be optionally dried.

The solid Form 4 of Cabozantinib salt with L-(+)-tartaric acid can be characterized by XRPD pattern having 2θ values 8.6°, 12.3°, 25.8° and 26.2° degrees 2 theta (±0.2 degrees 2 theta). The solid Form 4 can be also characterized by XRPD pattern having 2θ values 8.6°, 12.3°, 17.3°, 23.3°, 25.8° and 26.2° degrees 2 theta (±0.2 degrees 2 theta). The solid form can be further characterized by XRPD pattern described in the following table:

| Angle (2θ) | Intensity (%) |
|---|---|
| 8.6 | 51.5 |
| 8.9 | 26.3 |
| 11.4 | 15.4 |
| 12.1 | 84.3 |
| 12.3 | 100.0 |
| 13.5 | 12.3 |
| 14.3 | 37.0 |
| 14.6 | 19.0 |
| 16.4 | 20.3 |
| 16.7 | 26.9 |
| 17.3 | 43.3 |
| 17.8 | 24.8 |
| 18.2 | 31.5 |
| 18.7 | 16.7 |
| 19.1 | 23.7 |
| 19.5 | 19.6 |
| 20.0 | 29.2 |
| 20.2 | 29.7 |
| 20.9 | 32.9 |
| 21.4 | 22.5 |
| 21.8 | 23.1 |
| 22.0 | 30.6 |
| 22.7 | 22.3 |
| 22.9 | 24.6 |
| 23.3 | 42.5 |
| 24.0 | 24.2 |
| 24.8 | 17.1 |
| 25.0 | 17.1 |
| 25.8 | 53.6 |
| 26.2 | 60.3 |
| 26.8 | 21.5 |
| 27.6 | 16.4 |
| 28.9 | 17.5 |
| 30.1 | 12.6 |
| 30.4 | 15.2 |
| 32.6 | 10.0 |
| 34.7 | 11.4 |

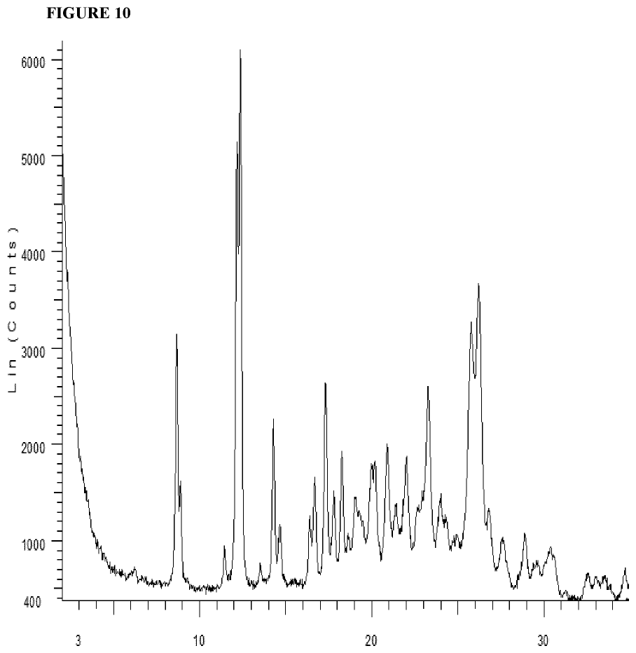

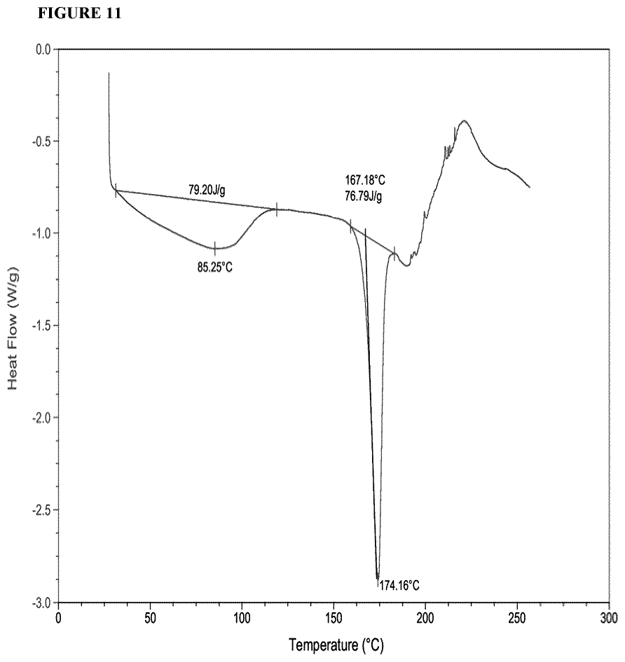

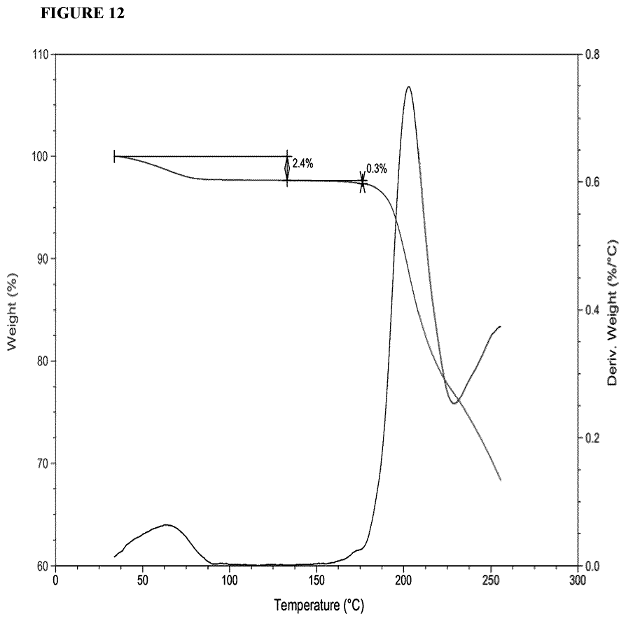

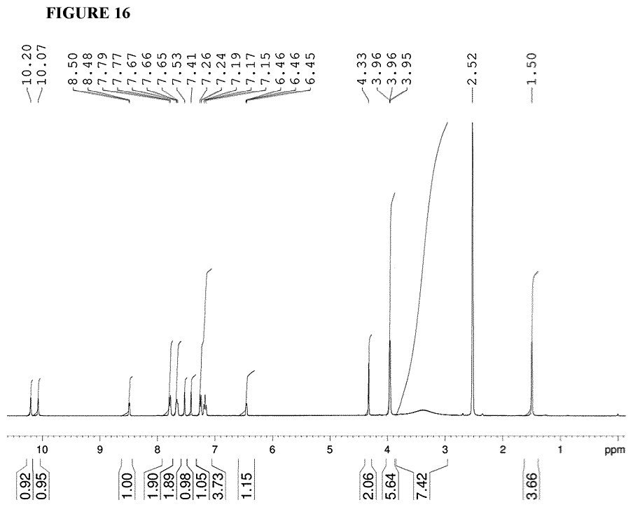

The solid Form 4 can be also characterized by XRPD pattern depicted in FIG. 10. The solid form can be further characterized by DSC pattern depicted in FIG. 11. The solid form can be further characterized by TGA pattern depicted in FIG. 12. The solid form can be also characterized by NMR pattern depicted in FIG. 16.

The solid Form 4 of Cabozantinib salt with L-(+)-tartaric acid can be prepared by a process comprising:
a. Mixing Cabozantinib with a mixture of anisol, butanol and 2.5-10% water (volume %) preferably at a temperature between 55° C. and 65° C.;
b. Adding L-(+)-tartaric acid;
c. Cooling the mixture preferably to a temperature between 20° C. and 25° C.;
d. Isolating the solid form.

The concentration of Cabozantinib in the mixture of anisol, butanol and water can be between 0.07 g/ml and 0.11 g/ml. The volume ratio between anisol and butanol can be between 0.8:1 and 1.2:1. 2.5-10% water (volume %) of water added to a mixture of anisol and butanol significantly improve the solubility of Cabozantinib in the mixture. The molar ratio between Cabozantinib and L-(+)-tartaric acid can be between 1:1 and 1:1.6, preferably between 1:1 and 1:1.2. Cabozantinib is mixed with the mixture of anisol, butanol and water at preferably a temperature between 55° C. and 65° C. To the mixture L-(+)-tartaric acid is added. L-(+)-tartaric acid can be added either as a solid or as a solution in a suitable solvent. The mixture is stirred at a temperature between 55° C. and 65° C. for between 20 and 60 minutes. The mixture is cooled preferably to a temperature between 20° C. and 25° C. and stirred at this temperature for between 8 and 15 hours to provide a suspension. The suspension is filtered off and obtained solid can be optionally dried.

The solid Form 2A of Cabozantinib salt with L-(+)-tartaric acid can be characterized by XRPD pattern having 2θ values 12.2°, 19.4°, 21.0°, 22.2°, 23.0° and 24.0° degrees 2 theta (±0.2 degrees 2 theta). The solid Form 2A can be also characterized by XRPD pattern having 20 values 12.2°, 18.5°, 19.4°, 21.0°, 22.2°, 23.0°, 24.0° and 31.4° degrees 2 theta (±0.2 degrees 2 theta). The solid form can be further characterized by XRPD pattern described in the following table:

| Angle (2θ) | Intensity (%) |
|---|---|
| 4.4 | 34.9 |
| 6.0 | 10.9 |
| 8.2 | 14.5 |
| 8.5 | 41.1 |
| 8.7 | 31.5 |
| 11.2 | 15.4 |
| 12.0 | 62.4 |
| 12.2 | 100.0 |
| 13.1 | 12.5 |
| 14.0 | 38.8 |
| 14.4 | 28.1 |
| 14.9 | 14.0 |
| 16.1 | 21.6 |
| 16.4 | 31.8 |
| 17.0 | 38.7 |
| 17.5 | 33.4 |
| 17.9 | 18.1 |
| 18.5 | 19.2 |
| 19.4 | 55.1 |
| 19.9 | 31.5 |
| 20.8 | 32.4 |
| 21.0 | 29.1 |
| 21.4 | 47.7 |
| 21.8 | 31.2 |
| 22.2 | 60.4 |
| 23.0 | 53.5 |
| 23.7 | 53.5 |
| 24.0 | 32.6 |
| 24.5 | 73.3 |
| 24.8 | 53.8 |
| 25.7 | 55.3 |
| 26.0 | 48.2 |
| 26.8 | 49.8 |
| 27.2 | 62.9 |
| 27.7 | 41.7 |
| 28.4 | 33.8 |
| 29.0 | 35.2 |
| 30.2 | 25.4 |
| 31.4 | 22.9 |
| 31.9 | 21.4 |
| 32.6 | 22.0 |
| 33.2 | 22.5 |
| 33.9 | 21.4 |
| 34.0 | 21.6 |

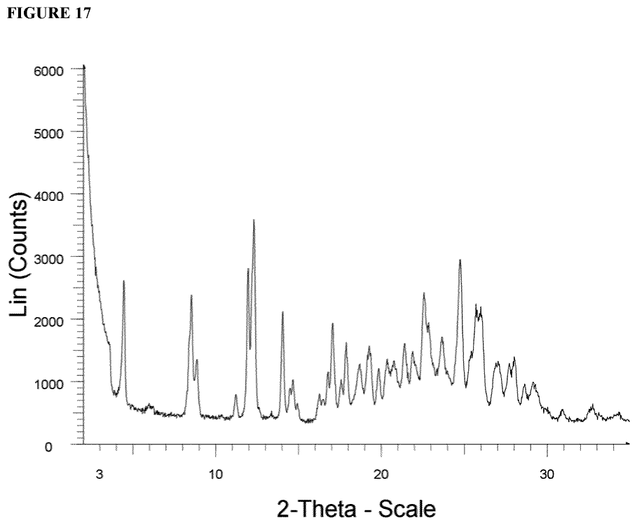

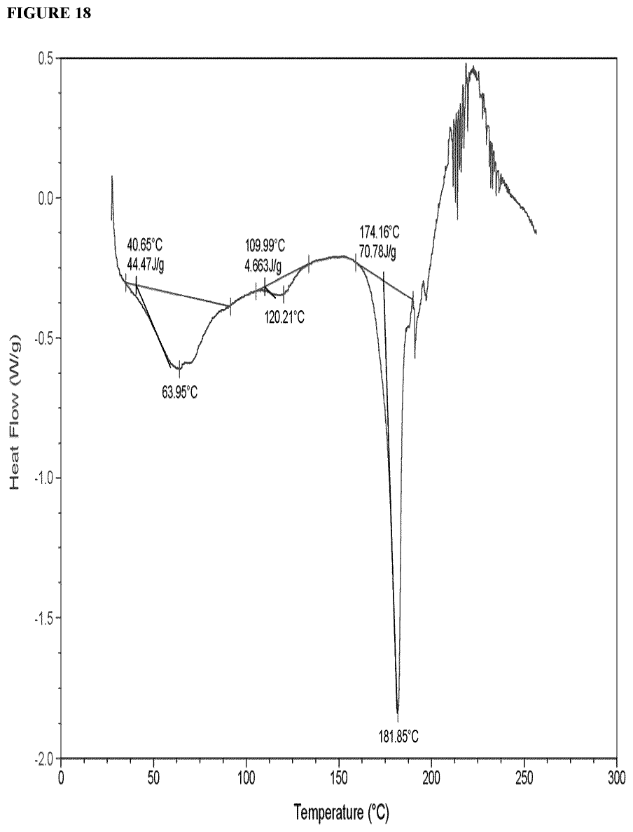

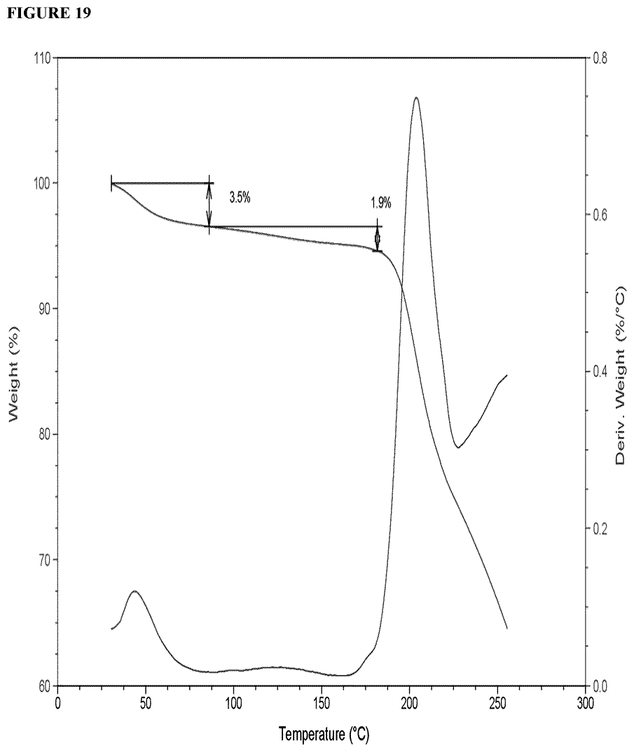

The solid Form 2A can be also characterized by XRPD pattern depicted in FIG. 17. The solid form can be further characterized by DSC pattern depicted in FIG. 18. The solid form can be further characterized by TGA pattern depicted in FIG. 19.

The solid Form 2A of Cabozantinib salt with L-(+)-tartaric acid can be prepared by a process comprising:
a. Mixing Cabozantinib with a mixture of tetrahydrofurane, butyl acetate and dimethylformamide to obtain Mixture 1;
b. Dissolving L-(+)-tartaric acid with methanol and butyl acetate and stirring the mixture preferably at a temperature between 40° C. and 60° C. to obtain Mixture 2;
c. Adding Mixture 1 into Mixture 2;
d. Stirring obtained mixture;
e. Cooling the mixture to a temperature between 10° C. and 30° C.;
f. Isolating the solid Form 2A.

The molar ration between Cabozantinib and L-(+)-tartaric acid can be between 1:1 and 1:1.6.

The concentration of Cabozantinib in the mixture of tetrahydrofurane, butyl acetate and dimethyl formamide can be between 0.1 g/g and 0.3 g/g. The ratio (wt/wt) between tetrahydrofurane, butyl acetate and dimethyl formamide can be between 1:0.7:0.3 and 1:1.5:0.8. Cabozantinib is mixed with a mixture of tetrahydrofurane, butyl acetate and dimethylformamide to obtain Mixture 1. The concentration of L-(+)-tartaric acid in methanol and butyl acetate can be between 0.08 g/g and 0.3 g/g. The ratio (wt/wt) between methanol and butyl acetate can be between 1:0.3 and 1:0.6. L-(+)-tartaric acid is mixed with a mixture of methanol and butyl acetate, the mixture is heated to a temperature between 40° C. and 60° C. and can be stirred at this temperature for between 30 and 180 minutes to obtain Mixture 2.

Mixture 1 is added into Mixture 2 at a temperature between 40° C. and 60° C. Mixture 1 can be added in several portions, for example in 1 or 2 or 3 or 4 or 5 or 6 portions. Obtained mixture was stirred at a temperature between 40° C. and 60° C. for between 2 and 10 hours. The mixture is then cooled to a temperature between 10° C. and 30° C. and stirred at this temperature for between 2 and 10 hours. Obtained suspension is filtered off and obtained solid can be washed with methanol heated to between 35° C. and 45° C. to provide solid Form 2A of Cabozantinib salt with L-(+)-tartaric acid.

The invention will be further described with reference to the following examples.

### EXAMPLES

Nuclear magnetic resonance spectroscopy (NMR) was performed using Avance III 400 MHz NMR spectrometer.
DCS patterns were obtained using the following conditions: 10° C./min->300° C.
XRPD spectrum was obtained using the following measurement conditions:
Panalytical Empyrean diffractometer with Θ/2Θ geometry (transmition mode), equipped with a PixCell 3D detector;

| Start angle (2θ): | 2.0° | |
|---|---|---|
| End angle (2θ): | 35.0° | |
| Step size: | 0.026° | |
| Scan speed: | 0.0955°/seconds | |
| Radiation type: | Cu | |
| Radiation wavelengths: | 1.5406Å (Kα1) | |
| Divergence slit: | ½° | |
| Antiscatter slit: | ½° | |
| Soller slit: | 0.02 | rad |
| Detector slit: | 7.5 | mm |
| Rotation speed: | 30 | rpm |

### Example 1: Cabozantinib Salt with L-(+)-Tartaric Acid, Form 1

2 g of Cabozantinib were mixed with a mixture of 20 ml of tetrahydrofurane and 0.6 ml of water at 60° C. The mixture was cooled to 40° C. and a mixture of 0.599 g of L-(+)-tartaric acid in 2 ml of tetrahydrofurane was added. The mixture was cooled to a temperature between 20° C. and 25° C. and stirred at this temperature for 24 hours. Obtained suspension was filtered off and obtained solid was dried under vacuum (10 kPa) at 40° C. for 24 hours to provide 1.4 g of solid Form 1 of Cabozantinib salt with L-(+)-tartaric acid.

### Example 2: Cabozantinib Salt with L-(+)-Tartaric Acid, Form 1

0.5 g of Cabozantinib was mixed with a mixture of 5 ml of tetrahydrofurane, 5 ml of ethylacetate and 0.15 ml of water. The mixture was heated to 60° C. and 0.15 g of L-(+)-tartaric acid was added. The mixture was left cooled to a temperature between 20° C. and 25° C. in the course of 10 hours. Obtained suspension was filtered and obtained solid was washed with a mixture of 1 ml of tetrahydrofurane and 1 ml of ethylacetate and dried on air for 12 hours to provide 0.622 g of solid Form 1 of Cabozantinib salt with L-(+)-tartaric acid.

### Example 3: Cabozantinib Salt with L-(+)-Tartaric Acid, Form 2

0.5 g of Cabozantinib was mixed with a mixture of 3 ml of tetrahydrofurane, 3 ml toluene and 0.5 ml water at 60°. To the mixture 0.15 g of L-(+)-tartaric acid was added. The mixture was stirred at 60° C. for 20 min and then was cooled to a temperature between 20° C. and 25° C. in the course of between 8 and 12 hours. Obtained suspension was filtered off and obtained solid was washed with 1 ml of toluene and dried on air for 12 hours to provide 0.64 g of solid Form 2 of Cabozantinib salt with L-(+)-tartaric acid.

### Example 4: Cabozantinib Salt with L-(+)-Tartaric Acid, Form 2

0.5 g of Cabozantinib was mixed with a mixture of 3.5 ml of tetrahydrofurane and 3 ml of methanol and the mixture was heated to reflux (bath 70° C.). To the mixture 0.15 g of L-(+)-tartaric acid was added. The mixture was cooled to a temperature between 20° C. and 25° C. for 12 hours. Obtained suspension was diluted with a mixture of 1 ml THF and 1 ml MeOH to improve filterability. Obtained suspension was filtered off. Obtained solid was washed with 1 ml of methanol MeOH and dried on air at 20-25° C. for 12 hours to provide solid Form 2 of Cabozantinib salt with L-(+)-tartaric acid in 88% yield.

### Example 5: Cabozantinib Salt with L-(+)-Tartaric Acid, Form 3

0.5 g of Cabozantinib was mixed with a mixture of 5 ml of tetrahydrofurane and 0.15 ml of water at 60° C. To the mixture 0.15 g of L-(+)-tartaric acid was added. The mixture was left to cooled to a temperature between 20° C. and 25° C. in the course of 10 hours. Obtained suspension was filtered off. Obtained solid was washed with 1 ml of tetrahydrofurane THF and dried on air at 20-25° C. for 12 hours to provide solid Form 3 of Cabozantinib salt with L-(+)-tartaric acid in 91% yield.

### Example 6: Cabozantinib Salt with L-(+)-Tartaric Acid, Form 3

0.5 g of Cabozantinib was mixed with a mixture of 3 ml of tetrahydrofurane, 3 ml of acetone and 0.15 ml of water at 60° C. To the mixture 0.15 g of L-(+)-tartaric acid was added. The mixture was left to cooled to a temperature between 20° C. and 25° C. for 10 hours. Obtained suspension was filtered off. Obtained solid was washed with mixture of 1 ml of tetrahydrofurane and 1 ml acetone and dried on air at 20-25° C. for 12 hours to provide 0.577 g of solid Form 3 of Cabozantinib salt with L-(+)-tartaric acid.

### Example 7: Cabozantinib Salt with L-(+)-Tartaric Acid, Form 3

0.5 g of Cabozantinib was mixed with a mixture of 3 ml of tetrahydrofurane, 3 ml of ethanol and 0.5 ml of water at 60° C. To the mixture 0.15 g of L-(+)-tartaric acid was added. The mixture was stirred at 60° C. for 30 minute and then left cooled to a temperature between 20° C. and 25° C. in the course of 10 hours. Obtained suspension was filtered off Obtained solid was washed with a mixture of 0.5 ml of tetrahydrofurane and 0.5 ml of ethanol and EtOH and dried on air at 20-25° C. for 12 hours to provide 0.61 g of solid Form 3 of Cabozantinib salt with L-(+)-tartaric acid.

### Example 8: Cabozantinib Salt with L-(+)-Tartaric Acid, Form 4

0.5 g of Cabozantinib was mixed with a mixture of 3 ml of anisol, 3 ml of butanol and 0.15 ml of water at 70° C. To the mixture 0.15 g of L-(+)-tartaric acid was added. It was stirred at 60° C. for 20 minutes and then it was cooled to ambient temperature in the course of 10 hours. Obtained suspension was filtered off. Obtained solid was washed with 1 ml of butanol and dried on at 20-25° C. air for 12 hours to provide 0.622 g of solid Form 4 of Cabozantinib salt with L-(+)-tartaric acid.

### Example 9: Cabozantinib Salt with L-(+)-Tartaric Acid, Form 2A and Form 2

3.8 g of L-(+)-tartaric acid was dissolved in 22 g of MeOH and 10 g of n-butyl acetate. The mixture was heated to 55° C. and stirred at this temperature for 60 minutes. To the mixture a mixture of 8.5 g of Cabozantinib with 20 g of tetrahydrofurane and 20 g of n-butyl acetate and 10 g of dimethylformamide was added at 55° C. Obtained mixture was stirred at 55° C. for 3 hours, cooled to 25° C. and stirred at 25° C. for 2 hours. Obtained suspension was filtered off, washed with 40 g of methanol heated to 40° C. to provide Cabozantinib salt with L-(+)-tartaric acid, Form 2A in 87% yield.

Obtained Form 2A was dried at 85° C. for 5 hours to provide Cabozantinib salt with L-(+)-tartaric acid Form 2 in quantitative yield.

### Example 10: Solubilities of Cabozantinib Salt with L-(+)-Tartaric Acid

Solubilities of Cabozantinib salt with L-(+)-tartaric acid were measured and are summarized in the following Table:

| | |
|---|---|
| Cabozantinib salt with | Solubility μg/ml |

| L-(+)-tartaric acid | pH 1.2 | pH 4.5 |
|---|---|---|
| Form 3 | 21 | 15 |
| Form 2 | 112 | 25 |

Samples were prepared by dissolving 12 mg of the sample in 50 ml of relevant solvent. Samples were mixed for 60 minutes at 37° C. and measured by HPLC method.

It can be concluded that solubilities of prepared solid forms of Cabozantinib salt with L-(+)-tartaric acid are improved in comparison with the solid form described in WO2010083414 application. Prepared solid forms of salts of Cabozantinib salt with L-(+)-tartaric acid show good crystallinity and low hygroscopicity.

1. A Cabozantinib salt with L-(+)-tartaric acid.

2. A solid form of the compound according to claim 1.

3. The solid form according to claim 2, which is Form 1, characterized by an XRPD pattern having 2θ values 3.9°, 13.5°, 14.8° and 23.1° degrees 2 theta (±0.2 degrees 2 theta).

4. The solid form according to claim 3, which is further characterized by an XRPD pattern having 2θ values 3.9°, 6.7°, 9.2°, 13.5°, 14.8°, 15.5° and 23.10 degrees 2 theta (0.2 degrees 2 theta).

5. The solid form according to claim 2, which is Form 2, characterized by an XRPD pattern having 2θ values 12.2°, 19.4°, 22.2° and 230 degrees 2 theta (±0.2 degrees 2 theta).

6. The solid form according to claim 5, which is further characterized by an XRPD pattern having 2θ values 4.4°, 8.5°, 12.2°, 14.0°, 19.4°, 22.2° and 23.0° degrees 2 theta (±0.2 degrees 2 theta).

7. A process of the preparation of the solid form according to claim 5, which comprises:
a) mixing Cabozantinib with a mixture of tetrahydrofurane and methanol;
b) adding L-(+)-tartaric acid;
c) cooling the mixture; and
d) isolating the solid form.

8. A process of the preparation of the solid form according to claim 5, which comprises drying a solid Form 2A of Cabozantinib salt with L-(+)-tartaric acid, wherein the solid Form 2A is characterized by an XRPD pattern having 2θ values 12.2°, 19.4°, 21.0°, 22.2°, 23.0° and 24.0° degrees 2 theta (±0.2 degrees 2 theta).

9. A process of the preparation of the solid form according to claim 5, which comprises:
a) mixing Cabozantinib with a mixture of tetrahydrofurane, butyl acetate and dimethylformamide to obtain Mixture 1;
b) dissolving L-(+)-tartaric acid with methanol and butyl acetate and stirring the mixture to obtain Mixture 2;
c) adding Mixture 1 into Mixture 2;
d) stirring the obtained mixture;
e) cooling the mixture;
f) isolating the solid Form 2A; and
g) drying the solid Form 2A.

10. The solid form according to claim 2, which is Form 3, characterized by an XRPD pattern having 2θ values 10.2°, 13.5°, 15.4° and 22.8° degrees 2 theta (±0.2 degrees 2 theta).

11. The solid form according to claim 10, which is further characterized by an XRPD pattern having 2θ values 3.9°, 8.4°, 10.2°, 12.8°, 13.5°, 15.4°, 22.8° and 25.9° degrees 2 theta (±0.2 degrees 2 theta).

12. The solid form according to claim 2, which is Form 4, characterized by an XRPD pattern having 2θ values 8.6°, 12.3°, 25.8° and 26.2° degrees 2 theta (±0.2 degrees 2 theta).

13. The solid form according to claim 12, which is further characterized by an XRPD pattern having 2θ values 8.6°, 12.3°, 17.3°, 23.3°, 25.8° and 26.2° degrees 2 theta (±0.2 degrees 2 theta).

14. The process according to claim 7, wherein said mixing step a) is carried out at a temperature between 60° C. and 70° C.; and said cooling step c) cools the mixture to a temperature between 20° C. and 25° C.

15. The process according to claim 9, wherein said dissolving step b) includes stirring the mixture at a temperature between 40° C. and 60° C.; and said cooling step e) cools the mixture to a temperature between 10° C. and 30° C.
